(19) United States
(12) Patent Application Publication
(10) Pub. No.: US 2023/0149269 A1
(43) Pub. Date: May 18, 2023

(54) OIL-IN-WATER TYPE COSMETIC COMPOSITION WITH VISIBLE OIL PARTICLES

(71) Applicant: MCTECH CO., LTD., Siheung-si (KR)

(72) Inventors: JEONG WOON CHI, Bucheon-si (KR); BEUM JUN KIM, Siheung-si (KR)

(73) Assignee: MCTECH CO., LTD., Siheung-si (KR)

(21) Appl. No.: 17/985,933

(22) Filed: Nov. 14, 2022

(30) Foreign Application Priority Data
Nov. 17, 2021 (KR) 10-2021-0158658

(51) Int. Cl. A61K 8/06; A61K 8/92; A61K 8/58; A61K 8/34; A61K 8/81; A61Q 17/04; A61Q 19/08

(52) U.S. Cl. CPC A61K 8/062; A61K 8/922; A61K 8/585; A61K 8/345; A61K 8/8147; A61K 8/8129; A61Q 17/04; A61Q 19/08; A61K 2800/33

(57) ABSTRACT

The present invention relates to an oil-in-water type cosmetic composition which includes an oil phase containing at least one selected from the group consisting of general oil, silicon oil and a gelling agent; and a water phase containing carbomer, PVA and purified water. An oil-in-water type cosmetic composition provided in the present invention contains oil particles including oil and water components, thereby improving visual aesthetic sense and maintaining the stability of emulsified particles even without any surfactant and emulsifier, so as to prevent a coalesce with other oils and also maximize an overall feeling of use, including spreadability and absorbency when applied to the skin due to low hardness. In addition, the composition does not contain any surfactant and emulsifier and thus minimize an irritation to the skin.

### BACKGROUND OF THE INVENTION

### 1. Field of the Invention

The present invention relates to an oil-in-water type cosmetic composition with visible oil particles, and more particularly, to an oil-in-water type cosmetic composition which contains oil particles including oil and water components, thereby maintaining the stability of emulsified particles even without any surfactant and emulsifier, improving aesthetic sense, and having the oil particles gently permeate when applied to the skin.

### 2. Description of the Prior Art

Cosmetics have a function of protecting the skin from external factors such as ultraviolet rays and environmental pollution and delaying aging. Most cosmetics are present in a state in which a water phase is dispersed in an oil phase or an oil phase is dispersed in a water phase. At this time, the use of an emulsifier (surfactant) is essential to maintain a stable dispersion state.

The emulsifier is a molecule having an amphiphilic structure consisting of a hydrophilic group which is easy to be compatible with water and a hydrophobic group which is easy to be compatible with oil. The emulsifier is located at an interface between the oil phase and the water phase so as to lower an interfacial tension, and forms an oil/water interface film at a phase boundary so as to prevent particles from coalescing with each other.

Emulsifiers may be classified according to various properties such as use, performance, chemical structure, etc. In general, the emulsifiers may be divided into ionic and nonionic characters according to their dissociation properties in water. Ionic emulsifiers which dissociate when dissolved in water may be further divided into anionic, cationic and amphoteric emulsifiers. A representative example of anionic emulsifiers may be a fatty acid salt soap, and that of cationic emulsifiers may be a quaternary ammonium compound. In addition, nonionic emulsifiers which do not dissociate into ions when dissolved in water may have various hydrophilic parts such as glycerol, polyglycerol, sorbitan, polyoxyethylene glycol, etc., and may have an ester bond or an ether bond with lipophilic parts such as fatty acid, fatty alcohol or the like.

Many commercially available emulsifiers may be used with an approval for safety. Nevertheless, the emulsifiers may cause an interfacial action even in skin cells and thus destroy a phospholipid layer, thereby causing an irritation or allergy in the skin. A loss of the lipid layer may weaken a barrier and increase skin permeability. The barrier with increased permeability may easily lose moisture, thus resulting in dull and dry skin. In addition, the penetration of foreign substances may be facilitated, thus resulting in a sensitive skin reaction such as an allergy.

However, when any surfactant and emulsifier is not included, the hardness of oil-in-water type oil particles may be lowered and the size of oil particles may increase due to growing possibility of coalescing with other oil particles other than a desired oil.

Thus, there is a need for an oil-in-water type cosmetic composition which can improve visual aesthetic sense while maintaining the stability of oil particles even without any separate surfactant and emulsifier, and also can maximize an overall feeling of use, including spreadability and absorbency, while minimizing an irritation when applied to the skin.

In a cosmetic field, a capsule is mainly used to prevent spoilage of specific ingredients. In the cosmetic field, cosmetics have been developed in such a way that the capsule per se is processed separately from a cosmetic composition and then mixed with the cosmetic composition, but the conventional method has problems of requiring a cumbersome preparation process and high preparation costs and also problems of leaving capsule remains on the skin after being applied thereto, thus causing a user to feel discomfort. Accordingly, there is a need to develop a cosmetic composition containing a stable capsule which can be automatically formed in the cosmetic without a separate capsule preparation process while keeping constant even when the cosmetics are stored for a long time.

In the present invention, a cosmetic composition containing oil particles has been prepared in a form in which an oil phase is dispersed in a water phase without a separate capsule preparation process and an effect of maintaining the stability of the emulsified particles even without any surfactant and emulsifier has been tested so as to complete the present invention.

### SUMMARY OF THE INVENTION

One object of the present invention is to provide an oil-in-water type cosmetic composition which contains oil particles including oil and water components, thereby maintaining the stability of emulsified particles even without any surfactant and emulsifier and having oil particles gently permeate when applied to the skin with improved aesthetic sense.

However, the technical objects to be achieved by the present invention are not limited to those mentioned above, and other objects that are not mentioned will be clearly understood by those skilled in the art from the following description.

According to one embodiment of the present invention, there may be provided an oil-in-water type cosmetic composition which contains: an oil phase including at least one selected from the group consisting of general oil, silicone oil, and a gelling agent; and a water phase including at least one selected from the group consisting of carbomer, polyvinyl alcohol (PVA) and purified water.

In the present invention, there may be provided an oil-in-water type cosmetic composition in which the oil phase contains 80 to 99% by weight of the general oil and the silicone oil, and 1 to 20% by weight of the gelling agent.

In the present invention, there may be provided an oil-in-water type cosmetic composition in which the water phase contains: 0.1 to 0.5% by weight of the carbomer; 0.1 to 2% by weight of the PVA; and a remainder of purified water.

In the present invention, there may be provided an oil-in-water type cosmetic composition in which the gelling agent contains at least one selected from the group consisting of dextrin palmitate ethylhexanoate (DPEH), dextrin myristate (DM), polyacrylate, dextrin and fatty acid esters, glycerol and fatty acid esters, polyamide, and glutamide.

In the present invention, the oil-in-water type cosmetic composition may be characterized by not containing any surfactant and emulsifier.

An oil-in-water type cosmetic composition provided in the present invention may contain oil particles including oil and water components, which can improve visual aesthetic sense while maintaining the stability of emulsified particles even without any surfactant and emulsifier, so as to prevent a coalescence with other oils, and also maximize an overall feeling of use, including spreadability and absorbency when applied to the skin due to low hardness.

### BRIEF DESCRIPTION OF THE DRAWINGS

FIG. 1 is a schematic view showing a device used for preparation in the present invention.

FIG. 2 is an image of formulations formed according to Examples 1 to 4 of the present invention.

FIGS. 3 to 6 are specific images of formulations formed according to Examples 1 to 4 of the present invention under a microscope (X40).

FIG. 7 is an image of formulations formed according to Comparative Examples 1 to 4 of the present invention.

FIGS. 8 to 11 are specific images of formulations formed according to Comparative Examples 1 to 4 of the present invention under a microscope (X40).

### DETAILED DESCRIPTION OF THE EXEMPLARY EMBODIMENTS

Hereinafter, the present invention will be described in detail through exemplary embodiments for better understanding of the present invention. However, the following exemplary embodiments are provided only for the purpose of illustrating the present invention, and thus the scope of the present invention is not limited thereto.

According to one embodiment of the present invention, there may be provided an oil-in-water type cosmetic composition which contains: an oil phase including at least one selected from the group consisting of general oil, silicone oil, and a gelling agent; and a water phase including at least one selected from the group consisting of carbomer, polyvinyl alcohol (PVA) and purified water.

In the present invention, the term “oil-in-water type” may refer to an emulsion in which oil droplets are dispersed in water, also called as an o/w type emulsion, and a representative example thereof may include milk, mayonnaise, varnishing cream, etc. In addition, a counterpart thereof may be a “water-in-oil type” emulsion, which means a turbid fluid or an emulsion, in which moisture is dispersed as fine particles in oil and thus is present in an emulsified state.

The general oil may be provided to change, dissolve, or suspend other substances in terms of physical properties. A suitable general oil may include: natural oils such as coconut oil; hydrocarbons such as mineral oil and hydrogenated polyisobutene; fatty alcohols such as octyldodecanol; esters such as C12-C15 alkyl benzoates; diesters such as propylene dipelargonate; and triesters, such as glyceryl trioctanoate, but is not limited thereto.

The general oil component may also be a mixture of oils with different viscosities.

An example of low-viscosity oils may include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, cocodicaprylate/caprate, decyl isostearate, isodecyl oleate, isodecyl neopentanoate, isohexyl neopentanoate, octyl palmitate, dioctyl malate, tridecyl octanoate, myristyl myristate, octyldodecanol, or mixtures of octyldodecanol, acetylated lanolin alcohol, cetyl acetate, isododecanol, polyglyceryl 3-diisostearate, or mixtures thereof.

An example of high-viscosity oils may include castor oil, lanolin and lanolin derivatives, triisocetyl citrate, sorbitan sesquioleate, C6-18 triglycerides, caprylic/capric/triglycerides, coconut oil, corn oil, cottonseed oil, glyceryl triacetyl hydroxystearate, glyceryl triacetyl ricinolate, glyceryl trioctanoate, hydrogenated castor oil, linseed oil, mink oil, olive oil, palm oil, rapeseed oil, soybean oil, sunflower seed oil, tallow, tricaprine, trihydroxystearin, triisostearin, trilaurine, trilinolein, trimyristine, triolein, tripalmitin, tristearin, walnut oil, wheat germ oil, cholesterol, or mixtures thereof. Among other optional non-silicone fatty substances, there may be mineral oils such as liquid paraffin or liquefied petroleum, animal oils such as perhydrosqualene or arara oil, or optionally vegetable oils such as sweet almond, calophyllum, palm, castor oil, avocado oil, jojoba oil, olive or cereal germ oil. In addition, esters of lanolic acid, oleic acid, lauric acid, stearic acid or myristic acid, for example alcohols such as oleyl alcohol, linoleyl or linolenyl alcohol, isostearyl alcohol or octyldodecanol, or acetylglycerides, octanoates, decanoates or ricinolates of alcohols or polyalcohols may be used.

The silicone oil may include a silicon oil or a mixture of silicon oils, which includes at least one selected from the group consisting of phenyltrimethione, dimethicone, cyclomethicone, polydimethylsiloxane, and silicone gum, or a mixture of silicon oils, and suitably phenyltrimethicone in terms of improving the percutaneous absorption and feeling of use of the oil-in-water type cosmetic composition.

In the present invention, there may be provided an oil-in-water type cosmetic composition, in which the oil phase includes 75 to 99% by weight and 80 to 95% by weight of the general oil and the silicone oil, and suitably 80 to 90% by weight thereof in terms of improving the percutaneous absorption and soft feeling of use of the oil-in-water type cosmetic composition, but is not limited thereto.

There may be provided an oil-in-water type cosmetic composition, in which the oil phase includes 40 to 60% by weight and 40 to 55% by weight of the general oil, and suitably 40 to 50% by weight thereof in terms of forming soft oil particles of the oil-in-water type cosmetic composition, but is not limited thereto.

There may be provided an oil-in-water type cosmetic composition, in which the oil phase includes 30 to 60% by weight and 30 to 55% by weight of the silicone oil, and suitably 30 to 50% by weight thereof in terms of improving the percutaneous absorption and soft feeling of use of the oil-in-water type cosmetic composition, but is not limited thereto.

In the present invention, there may be provided an oil-in-water type cosmetic composition, in which the oil phase includes 0.1 to 25% by weight and 5 to 20% by weight of the gelling agent, and suitably 10 to 20% by weight thereof in terms of lowering the hardness of particle surface of the oil, preventing coalescence with other oil particles, and gelling only oils other than silicone oil, but is not limited thereto.

The gelling agent may maintain the stability of the oil phase particles, and form oil particles by gelling only oils other than silicone oil, thereby improving the feeling of use and aesthetic sense. However, departing from the above range, the strength of the oil particles may be remarkably low or high, and thus the feeling of use and aesthetic sense may be deteriorated when applied to the skin.

In the present invention, there may be provided an oil-in-water type cosmetic composition, in which the water phase includes 0.01 to 0.5% by weight and 0.1 to 0.5% by weight of the carbomer, and suitably 0.1 to 0.3% by weight thereof in terms of maintaining the stability of the oil-in-water type cosmetic composition, but is not limited thereto.

The carbomer may be an anionic polymer, in which an acidic high molecular compound is mainly polymerized in acrylic acid, and may have an effect of increasing the viscosity of the disclosed oil-in-water type cosmetic composition to maintain stability, and outside the above range, aesthetic sense may be deteriorated when applied to the skin.

In the present invention, there may be provided an oil-in-water type cosmetic composition, in which the water phase includes 0.01 to 2% by weight and 0.1 to 2% by weight of the polyvinyl alcohol (PVA), and suitably 0.1 to 1% by weight thereof in terms of increasing an emulsifying capacity of the oil-in-water type cosmetic composition and preventing coalescence between particles during a preparation process of the oil-in-water cosmetic type composition, but is not limited thereto.

The polyvinyl alcohol (PVA) may be a water-soluble material, and may form a colloidal liquid when dispersed in water, thereby providing an effect of having excellent dispersion stability. When mixed with the carbomer and water-soluble silicone used in the present invention, the hardness of the surface of oil particles may become too low due to a decrease in the content of the gelling agent, thus causing a problem of coalescence between the particles. However, the use of the PVA may contribute to improving the dispersibility and emulsification of oil particles. However, outside the above range, water resistance may be reduced, thus causing a decrease in the feeling of use and aesthetic sense when applied to the skin.

In the present invention, there may be provided an oil-in-water type cosmetic composition, in which the water phase further includes 0.001 to 0.7% by weight and 0.1 to 0.6% by weight of water-soluble silicone, and suitably 0.1 to 0.5% by weight thereof in terms of improving adhesion when applied to the skin, but is not limited thereto.

The water-soluble silicone may have a wax-like property at room temperature, have excellent compatibility with water, and serve to increase an adhesion to the skin by gradually increasing water. There may be provided an oil-in-water type cosmetic composition which contains at least one selected from the group consisting of amodimethicone, dimethicone/vinyl dimethicone crosspolymer and BIS-PEG-18 methyl ether dimethyl silane, and specifically an oil-in-water type cosmetic composition which contains suitably amodimethicone in terms of increasing an emulsification capacity of the oil-in-water type cosmetic composition by increasing a water phase during mixed emulsification of the oil phase and the water phase in the oil-in-water type formulation, but is not limited thereto.

In the present invention, there may be provided an oil-in-water type cosmetic composition, in which the water phase contains a remainder of purified water.

The type of solvent which may be further added to the oil-in-water type cosmetic composition may include, for example, water, saline, DMSO, or a combination thereof, and suitably purified water, but is not be particularly limited thereto. In addition, if necessary, the cosmetic composition may include a whitening agent, a moisturizer, a vitamin, a sunscreen, a perfume, a dye, an antibiotic, an antibacterial agent, and an antifungal agent.

In the present invention, there may be provided an oil-in-water type cosmetic composition in which the gelling agent contains at least one selected from the group consisting of dextrin palmitate ethylhexanoate (DPEH), dextrin myristate (DM), polyacrylate, dextrin and fatty acid esters, glycerol and fatty acid esters, polyamide, and glutamide, and suitably dextrin palmitate ethylhexanoate (DPEH) and dextrin myristate (DM) in terms of maintaining the stability of the oil phase particles and forming oil particles by gelling only oils other than silicone oil, but is not limited thereto.

The polyamide used herein may include a commercially available raw material, oleocraft, and the glutamide used herein may include a commercially available raw material, indiogel, including dibutyl ethylhexanoyl glutamide and dibutyl lauroyl glutamide.

In the present invention, the oil-in-water type cosmetic composition may be characterized by not containing any surfactant and emulsifier.

Since the oil-in-water type cosmetic composition does not contain any surfactant and an emulsifier, thus it is suitable to use the carbomer as a thickener, and departing from the scope of the present invention, the stability and application power of the formulation cannot be increased. In addition, the oil-in-water type cosmetic composition may not cause any problems such as skin troubles, etc., due to the use of surfactants and emulsifiers.

In the present invention, the oil-in-water type cosmetic composition may take the form of a solution, an emulsion, a viscous mixture, and the like, and may be prepared in the form of lotion, nourishing lotion, nourishing essence, massage cream, beauty bath additive, body lotion, body milk, bath oil, baby oil, baby powder, shower gel, shower cream, sunscreen lotion, sunscreen cream, suntan cream, skin lotion, skin cream, sunscreen cosmetics, foundation, makeup base, BB cream, skin cover, concealer, lipstick, lip gloss, lip balm, face powder, two-way cake, eye shadow, eyeliner, mascara, cheek color, eyebrow pencils, cleansing milk, depilatory agent (makeup), face and body lotion, face and body cream, skin whitening cream, hand lotion, hair lotion, cosmetic cream, jasmine oil, bath soap, water soap, beauty soap, shampoo, hand sanitizer (hand cleaner), medicinal soap {non-medical use}, cream soap, facial wash, whole body cleaner, scalp cleaner, hair conditioner, cosmetic soap, tooth whitening gel, toothpaste, etc. For this purpose, the oil-in-water type cosmetic composition may further include a solvent, an appropriate carrier, an excipient or a diluent, which is commonly used in the preparation of a cosmetic composition within a range which does not impair the purpose and effect of the oil-in-water type cosmetic composition of the present invention.

The oil-in-water type cosmetic composition may maintain basic physical properties and quality in addition to the above-mentioned composition, if necessary, and may include ingredients widely known to and used by those skilled in the art as any conventional ingredient for preparation of each formulation.

In addition, the composition may include any UV protection ingredients, whitening ingredients, anti-wrinkle functional ingredients, antioxidant ingredients, moisturizing ingredients, antibacterial ingredients, etc., which may be widely known to and used by those skilled in the art, as a functional ingredient which can effectively act on the skin while enhancing affinity with the skin within a range which does not impair the purpose and effect of the oil-in-water type cosmetic composition of the present invention.

Furthermore, the oil-in-water type cosmetic composition of the present invention may form drops by using a membrane emulsifying device for preparing microemulsion according to the present applicant's Korean Patent Registration Publication No. 10-1868854 or a membrane emulsifying device for preparing coarse particles having a circulation tank of Korean Patent Registration Publication No. 10-1932005.

Hereinafter, the present invention will be described in more detail through exemplary embodiments. These embodiments are provided only for the purpose of illustrating the present invention in more detail, and thus it will be apparent to those skilled in the art that the scope of the present invention is not limited thereto according to the gist of the present invention.

### EXAMPLE

### Preparation Example

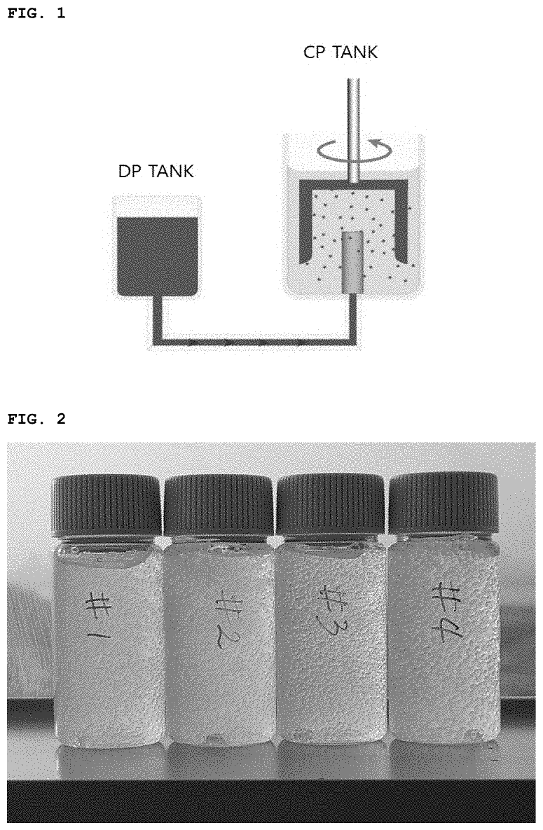

While maintaining an overall temperature of an apparatus as shown in FIG. 1 at 70 to 90° C., a DP tank was filled with an oil phase and a CP tank was filled with a water phase 1, and then the DP tank was pressurized with nitrogen or compressed air. As the oil phase (oil) in the DP tank was discharged to the water phase in the CP tank through pores of a membrane connected to an inside of the CP tank, oil drops of a certain size were formed and the oil phase in the DP tank was completely discharged. Then, the apparatus was cooled, a water phase 2 (alkali solution such as arginine, toromethamine, NaOH, etc.) was added to the CP tank to thicken a carbomer, thereby maintaining and stabilizing a dispersed state of oil drops.

Efficacy Evaluation According to Content Range of Oil Phase and Water Phase

TABLE 1

| | | | | | | Com- | Com- | Com- | Com- |
|---|---|---|---|---|---|---|---|---|---|
| | | | | | | parative | parative | parative | parative |
| | | Example | Example | Example | Example | Example | Example | Example | Example |
| Component | | 1 | 2 | 3 | 4 | l | 2 | 3 | 4 |
| Oil | Phenyl | 35 | 35 | 35 | 21 | 0 | 0 | 3 | 6 |
| phase | trimethicone | | | | | | | | |
| (g) | MCT | 28 | — | 14 | 17.5 | 27 | — | 12 | 10.5 |
| | OIL | | | | | | | | |
| | Jojoba | — | 28 | 14 | 17.5 | — | 27 | 12 | 10.5 |
| | oil | | | | | | | | |
| | DPEH | 7 | 7 | 7 | 14 | 3 | 3 | 3 | 3 |
| Water | Purified | 229.63 | 229.63 | 229.63 | 229.63 | 230.98 | 230.98 | 229.63 | 229.63 |
| phase | water | | | | | | | | |
| 1 | Propanediol | 13.5 | 13.5 | 13.5 | 13.5 | 13.5 | 13.5 | 13.5 | 13.5 |
| (g) | 1,2- | 5.4 | 5.4 | 5.4 | 5.4 | 5.4 | 5.4 | 5.4 | 5.4 |
| | hexanediol | | | | | | | | |
| | 1,3- | 13.5 | 13.5 | 13.5 | 13.5 | 13.5 | 13.5 | 13.5 | 13.5 |
| | butylene | | | | | | | | |
| | glycol | | | | | | | | |
| | Carbomer | 0.54 | 0.54 | 0.54 | 0.54 | 0.54 | 0.54 | 0.54 | 0.54 |
| | PVA | 1.35 | 1.35 | 1.35 | 1.35 | 0 | 0 | 1.35 | 1.35 |
| | EDTA | 0.14 | 0.14 | 0.14 | 0.14 | 0.14 | 0.14 | 0.14 | 0.14 |
| Water | Purified | 5.4 | 5.4 | 5.4 | 5.4 | 5.4 | 5.4 | 5.4 | 5.4 |
| phase | water | | | | | | | | |
| 2 | Arginine | 0.54 | 0.54 | 0.54 | 0.54 | 0.54 | 0.54 | 0.54 | 0.54 |
| (g) | | | | | | | | | |

As shown in above table 1, an oil-in-water type cosmetic composition containing each component of Examples 1 to 4 and Comparative Examples 1 to 4 was prepared according to the above preparation example, and then a particle state and feeling of use were evaluated and shown in table 2 below.

Specifically, as for the particle state of the oil-in-water type cosmetic composition prepared with the composition of above table 1, a degree of particle formation was visually observed in such a way that ⊚ indicates a formation of very stable particles, o indicates a formation of stable particles, Δ indicates a formation of non-uniform particles, and X indicates that particles are broken. In addition, the feeling of use was expressed by evaluating the strength of the oil particles on a scale of 1 to 5 in such a way that 1 indicates softness enough to hardly feel the particles when applied on the back of the hand, 2 indicates softness with a stable feel of particles, 3 indicates an average feel of particles, 4 indicates a hard feel of particles, and 5 indicates a very hard feel enough to spread on the skin only after being applied several times.

TABLE 2

| | | | | | Com- | Com- | Com- | Com- |
|---|---|---|---|---|---|---|---|---|
| | | | | | parative | parative | parative | parative |
| | Example | Example | Example | Example | Example | Example | Example | Example |
| Results | 1 | 2 | 3 | 4 | 1 | 2 | 3 | 4 |
| Particle | ◯ | ◯ | ◯ | ⊚ | X | X | X | Δ |
| state | | | | | | | | |
| (Appearance) | | | | | | | | |
| Feeling | 2 | 2 | 2 | 5 | 1 | 1 | 1 | 1 |
| of use | | | | | | | | |
| (Strength) | | | | | | | | |

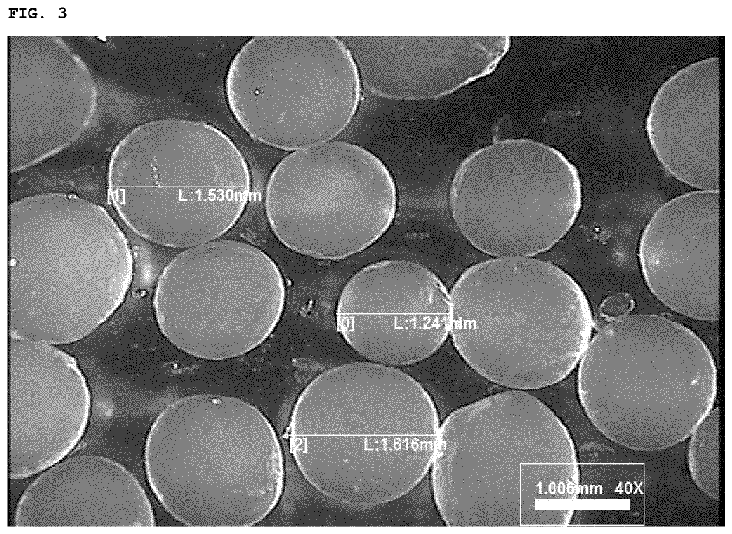

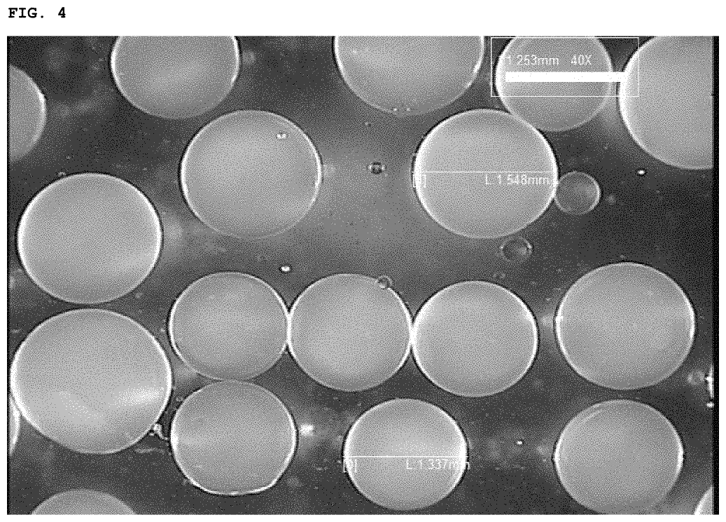

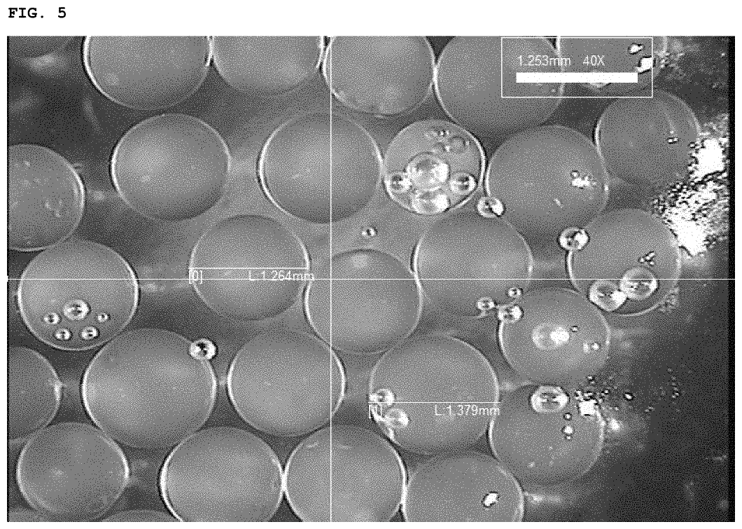

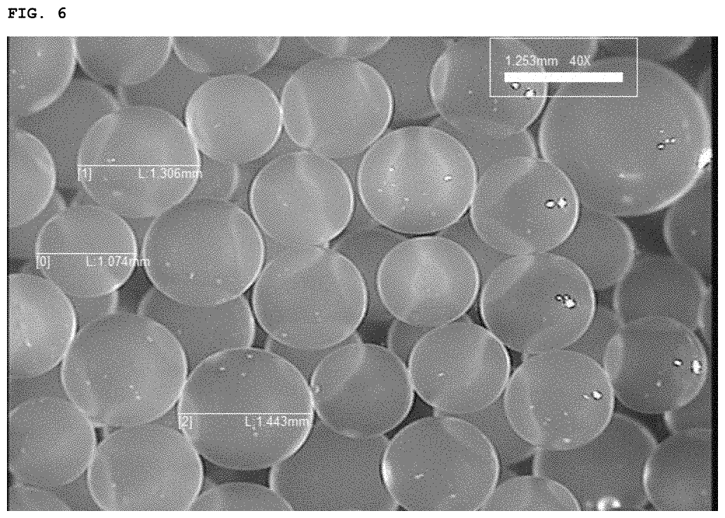

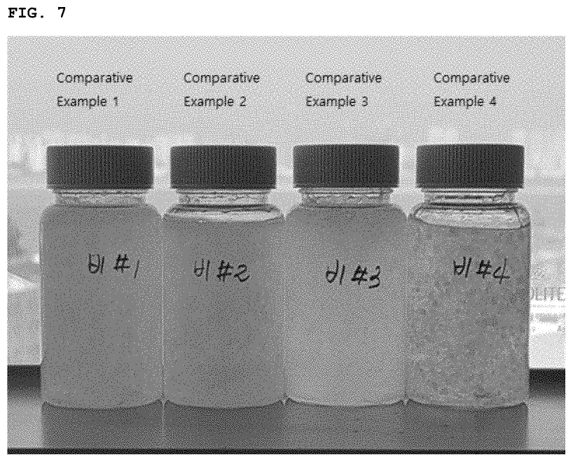

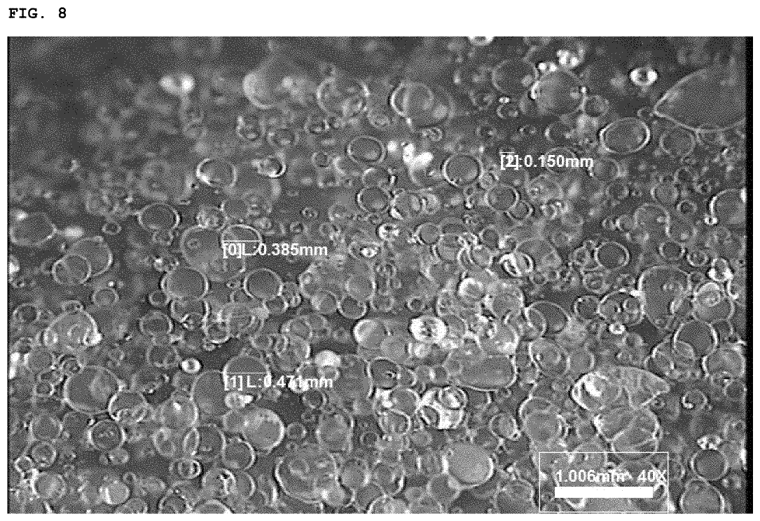

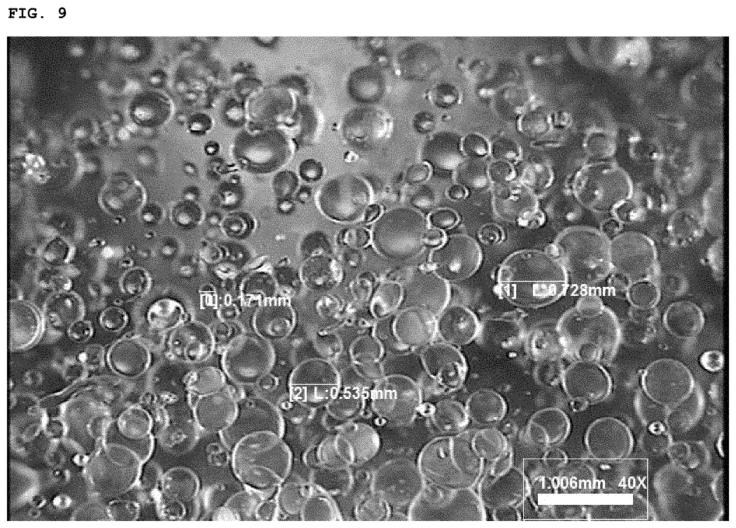

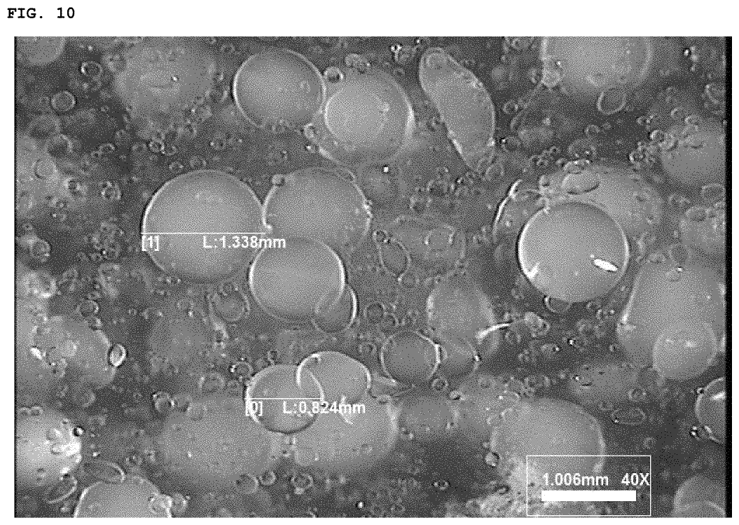

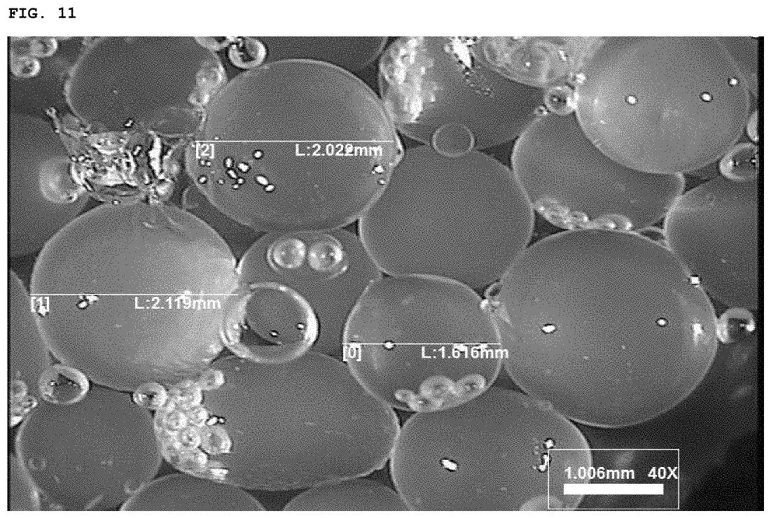

As shown in above table 2 and FIGS. 2 to 11, it could be confirmed from Examples 1 to 4 that the state of particles is all stably formed with the feeling of use being 2 or more, and it could be confirmed from Comparative Examples 1 to 4 that the state of particles is not generally stable with the feeling of use being all 1.

Specifically, Examples 1 to 3 showed a case in which the content of phenyl trimethicone, a silicone oil, is fixed to 50% by weight of the oil phase; DPEH, a gelling agent, is fixed to 10% by weight of the oil phase; and the water phases 1 and 2 are fixed at the same content with only the type of oil included in the oil phase changed. Example 4 shows a case in which the content of the silicone oil is lowered to 30% by weight of the oil phase and the content of the gelling agent is increased to 20% by weight of the oil phase, as compared with Examples 1 to 3.

Referring to above table 2 and FIGS. 2 to 6, it can be seen that particles can be stably generated in the oil which may be thickened regardless of MCT OIL and jojoba oil, which are types of gelling oil, and it can be seen that the uniformity and strength of the oil particles increase as the ratio of the gelling agent increases, and the feeling of use becomes excellent as the ratio of silicone oil increases. Accordingly, it could be seen that, suitably when included in the proportion of the oil phase according to the present invention, the particles of the oil are uniformly formed to a desired size or more, thereby improving the aesthetic sense and improving the feeling of use.

In addition, in Comparative Examples 1 and 2, in which silicone oil and PVA are not added, it could be confirmed that oil particles are broken into very small particles and a particle size becomes non-uniform in the CT tank discharge step due to low strength of oil particles regardless of the type of oil contained in the oil phase.

Furthermore, in Comparative Example 3, in which silicon oil is added in an amount of 10% by weight of the oil phase along with the PVA, it could be confirmed that oil particles are formed, but the coalesced particles are increased and floated toward a water surface, and the particle size becomes non-uniform with an elapse of time.

Moreover, in Comparative Example 4, in which silicon oil is added in an amount of 20% by weight of the oil phase along with the PVA, it could be confirmed that the oil particles are likely to be formed, but the particle size becomes non-uniform, thus making it impossible to commercialize.

Referring to above table 2 and FIGS. 7 to 11, when the ratio of silicone oil and PVA departs from the scope of the present invention, it can be seen that oil particles are coalesced or formed non-uniformly, making it difficult to prepare as an oil-in-water type cosmetic composition.

Accordingly, it could be confirmed that, suitably when silicone oil and PVA are added in the proportions according to the present invention, the oil particles are uniformly formed without coalescing during a preparation process of the cosmetic composition.

Thus, it could be confirmed in the case of preparing with the blending ratio of the present invention that it is possible to prepare an oil-in-water type cosmetic composition with improved aesthetic sense with visible particles by forming uniform particles without coalescing with each other, and the total oil particles become soft and excellent enough to prepare a cosmetic composition with excellent feeling of use, thus providing critical significance.

1. An oil-in-water type cosmetic composition, which comprises: an oil phase containing at least one selected from the group consisting of general oil, silicon oil and a gelling agent; and
a water phase containing at least one selected from the group consisting of carbomer, polyvinyl alcohol (PVA) and purified water.

2. The oil-in-water type cosmetic composition of claim 1, wherein the oil phase comprises 80 to 99% by weight of the general oil and the silicone oil, and 1 to 20% by weight of the gelling agent.

3. The oil-in-water type cosmetic composition of claim 1, wherein the water phase comprises: 0.1 to 0.5% by weight of the carbomer; and 0.1 to 2% by weight of the PVA.

4. The oil-in-water type cosmetic composition of claim 1, wherein the gelling agent comprises at least one selected from the group consisting of dextrin palmitate ethylhexanoate (DPEH), dextrin myristate (DM), polyacrylate, dextrin and fatty acid esters, glycerol and fatty acid esters, polyamide, and glutamide.

5. The oil-in-water type cosmetic composition of claim 1, wherein any surfactant and emulsifier is not comprised.
